(19) United States
(12) Patent Application Publication
(10) Pub. No.: US 2023/0043748 A1
(43) Pub. Date: Feb. 9, 2023

(54) DEVICE FOR DISPENSING A FLUID PRODUCT

(71) Applicant: APTAR FRANCE SAS, Le Neubourg (FR)

(72) Inventors: Mickaël BAILLIE, Acquigny (FR); Maxime HUPPÉ, Caudebec Les Elbeuf (FR); Mathieu NORRANT, Houlbec Cocherel (FR)

(73) Assignee: APTAR FRANCE SAS, Le Neubourg (FR)

(21) Appl. No.: 17/786,967

(22) PCT Filed: Dec. 15, 2020
(86) PCT No.: PCT/FR2020/052456

§ 371 Date: Jun. 17, 2022

(30) Foreign Application Priority Data
Dec. 19, 2019 (FR) FR1915029

(51) Int. Cl. A61M 15/00

(52) U.S. Cl. CPC A61M 15/0036; A61M 2205/8206; A61M 2205/3584; A61M 2205/33; A61M 2202/064

(57) ABSTRACT

A device for dispensing a fluid product, having a body (1) and a dispensing head (2) with a dispensing orifice (3) and axially movable. The body receives a vessel (10) containing one or two doses of product. The device has an electronic module (100) with a wireless communication module (101), a geographical location module (102), and a power source (103). The device has a sensor system (201, 202) for detecting and automatically signalling an attempt to use the device, the sensor system having a first sensor (201) arranged on the dispensing head (2) and a second sensor (202) on the body, the sensor system detecting handling representative of an attempt to use the device when the first and second sensors (201, 202) are activated simultaneously, the wireless communication module designed to make an automatic emergency call when the sensor system detects handling representative of an attempt to use the device.

The present invention relates to a device for dispensing a fluid product, and more particularly to a device of the single-dose or two-dose type that is adapted to dispense one or two doses of fluid in a single actuation.

Devices of the single-dose or two-dose type are well known. They generally comprise a vessel containing one or two doses of fluid product, and a dispensing head provided with a dispensing orifice, and which is axially movable with respect to said vessel during actuation. Documents EP0546607 and WO2016097603 describe devices of this type.Documents EP0546607 and WO2016097603 describe devices of this type.

For certain sensitive drugs, using the device and therefore dispensing a dose of the drug can indicate an emergency situation. For example, in the case of Naloxone-based drugs, which are in particular used to recover from an overdose (for drug addicts, but also for “normal” opioid users), the duration of activity of the drug is very limited, hence the necessity to rapidly consult a doctor or similar to obtain another dose once necessary. Another example relates to epinephrine, where it is also important that the patient goes rapidly to hospital for monitoring, as the effectiveness of epinephrine is only a few hours. This can be the same with other drugs, such as for example, Fentanyl.

Documents WO2008091838, US2016193408 and WO0193926 describe devices which detect the actuation of a device for dispensing drug, and which are capable of sending an alert, for example to an emergency call number. These † Translation of the title as established ex officio. devices allow to alert when the device is used, but if this use has been successful, and if the dose has been correctly dispensed, the alert to an emergency call number is, in reality, not useful. Such an alert is however highly desirable, if the user attempts to actuate the device, but is not successful in doing so, for example by lack of force, loss of knowledge, or malfunction of the device.

Documents CN102626533, US2017257436, WO2013154954 and US2009128330 describe other devices of the state of the art.

An object of the present invention is to provide a device for dispensing a fluid product, in particular of the single-dose or two-dose type, that does not have the above-mentioned drawbacks.

The present invention also aims to provide a device for dispensing a fluid product, in particular of the single-dose or two-dose type, which automatically calls an emergency number during an attempt to actuate the device.

Another object of the present invention is to provide a device for dispensing a fluid product, in particular of the single-dose or two-dose type, that is simple and inexpensive to manufacture and to assemble.

The present invention thus provide a device for dispensing a fluid product comprising a body and a dispensing head provided with a dispensing orifice and axially movable with respect to said body during actuation, said body receiving a vessel containing one or two doses of fluid product, said device comprising an electronic module which comprises a wireless communication module, such as a GSM module and/or a Wi-Fi module and/or a Bluetooth® module, a geographical location module, such as a GPS module and/or an antenna array, and a power source, such as a battery, said device comprising a sensor system for automatically detecting and signalling handling representative of an attempt to use said device, said sensor system having at least one first sensor arranged on said dispensing head and at least one second sensor arranged on said body, said sensor system detecting handling representative of an attempt to use said device when said first and second sensors are activated simultaneously, said wireless communication module being designed to make an automatic emergency call to an emergency number when said sensor system detects handling representative of an attempt to use said device.

Advantageously, said at least one first sensor is a microcurrent sensor, a capacitive sensor, an infrared sensor, a luminosity sensor, an audio sensor, a temperature sensor or a humidity sensor.

Advantageously, said at least one second sensor is a microcurrent sensor, a capacitive sensor, an infrared sensor, a luminosity sensor, an audio sensor, a temperature sensor or a humidity sensor.

Advantageously, before actuation, said electronic module is switched off or in standby mode, with a zero or minimum energy consumption.

Advantageously, said electronic module is switched on or awakened by said sensor system to go from its standby or switched off mode to an active mode.

Advantageously, said dispensing head includes a radial flange on which the user places one or more fingers during actuation, said at least one first sensor being disposed on said radial flange.

Advantageously, said body comprises a distal axial wall on which the user places one or more fingers, typically the thumb, during actuation, said at least one second sensor being disposed on said distal axial wall.

Advantageously, said electronic module comprises timeout means to postpone the automatic emergency call by a few seconds from detecting an attempt to use by the sensor system.

Advantageously, said vessel comprises a hollow tube having a proximal axial opening that is closed by a stopper that is adapted to slide in leaktight manner in said tube during actuation.

Advantageously, said dispensing head comprising a hollow cannula connected on one side to said dispensing orifice and provided on the other side with a perforator tip provided with an opening for piercing said stopper.

According to a first advantageous variant, said fluid product is a liquid.

According to a second advantageous variant, said fluid product is a powder.

Advantageously, said vessel contains one single dose of fluid product, dispensed in one single actuation.

In a variant, said vessel contains two doses of fluid product, dispensed in two successive actuations.

These and other characteristics and advantages will appear more clearly from the following detailed description made with reference to the accompanying drawings given by way of non-limiting examples, and wherein:

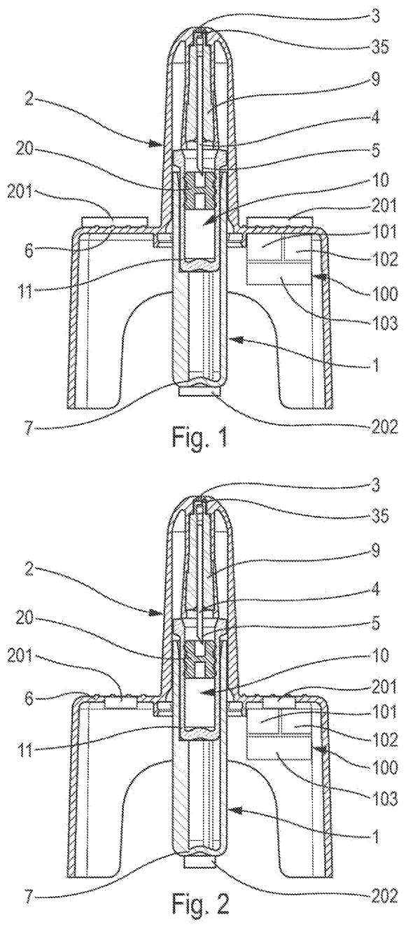

FIG. 1 is a schematic transverse cross-sectional view of a device of the single-dose type according to a first advantageous embodiment, in the rest position; and

FIG. 2 is a schematic view, similar to the view in FIG. 1 of a device of the single-dose type according to an advantageous second embodiment.

More specifically, the present invention relates to, on the one hand, a device of the single-dose type, such as for example, that disclosed in document EP0546607, and on the other hand, a device of the two-dose type, such as for example, that disclosed in document WO2016097603.

It is however understood that the present invention is not limited to these types of device, but is, however, applicable to all types of devices for dispensing fluid product, of the single-dose or two-dose type.

In the description, the terms “axial” and “radial” are relative to the longitudinal axis of the device. The terms “proximal” and “distal” are relative to the dispensing orifice 3 formed in the dispensing head 2.

The invention applies, on the one hand, to devices of the single-dose type, such as those represented in the figures, in which all of the dose of fluid product contained in the device is dispensed in one single actuation of the device, and on the other hand, to devices of the two-dose type, in which the fluid product contained in the device is dispensed in two successive actuations of the device.

The device of the single-dose type represented in figures comprises a body 1 which receives a vessel 10 containing a dose of fluid product, and a dispensing head 2, provided with a dispensing orifice 3, which is axially movable with respect to said body 1 during actuation.

Advantageously, said vessel 10 is formed by a blind hollow tube 11, e.g. made of glass, having a proximal axial opening 12 that is closed by a stopper 20, e.g. made of elastomer, that is adapted to slide in leaktight manner in said tube 11 during actuation.

The dispensing head 2 generally includes a cannula or needle 4 of generally cylindrical shape that is connected at one end to said dispensing orifice 3, and that is provided at its other end with a perforator tip 5 that is adapted to perforate said stopper 20 during actuation, the stopper 20 thus being moved in said vessel 10 so as to expel the dose(s) of fluid through said cannula 4, towards said dispensing orifice 3. The cannula 4 may be inserted into a cannula support 9 that may itself be fastened in said dispensing head 2. Advantageously, a spray profile 35 may be formed directly upstream from the dispensing orifice 3, e.g. between the end wall of said dispensing head 2 and the proximal axial end of said cannula support 9.

The dispensing head 2 comprises a radial flange 6 on which the use places one or more fingers during actuation.

Typically, the actuation of the device is done by placing one or more fingers on said radial flange 6, and one finger, typically the thumb, on a distal axial wall 7 of the body 1, and by axially moving the body 1 with respect to the dispensing head 2.

The device moreover comprises an electronic module 100 connected to a sensor system 201, 202.

The electronic module 100 advantageously comprises a wireless communication module 101, such as a GSM module, a geographical location module 102, such as a GPS module, and a power source 103, such as a battery.

At rest, the electronic module 100 is advantageously in standby mode, with a minimum energy consumption. In a variant, it could be completely switched off at rest, with therefore zero energy consumption. Awakening said electronic module is preferably done by the sensor system.

The sensor system comprises at least two sensors 201, 202 intended to detect a handling of the user representative of a clear desire to use the device. Thus, the present invention does not detect the actuation of the device, or the simple opening of the covering, but only a handling such as done when it is sought to actuate the device.

According to the invention, the sensor system includes at least one first sensor 201 disposed on the radial flange 6 of the dispensing head 2. In the embodiment shown, there are two first sensors 201 that are diametrically opposed on said radial flange 6. This at least one first sensor 201 detects the finger(s) that the user places on said radial flange 6 when it is sought to actuate the device. Said at least one first sensor 201, according to its structure and its operation, can be fixed on the radial flange 6, as illustrated in FIG. 1, or inserted in said radial flange 6, as illustrated in FIG. 2.

The sensor system also includes at least one second sensor 202 disposed in the distal axial wall 7 of the body 1. In the example shown, there is only one second sensor 202. This at least one second sensor 202 detects the finger(s), typically the thumb, that the user places on said distal axial wall 7 when it is sought to actuate the device. Said at least one second sensor 201, according to its structure and its operation, can also be fixed on the distal axial wall 7, as illustrated in FIG. 1. It could also be inserted into said distal axial wall 7.

The association of at least one first sensor 201 on the radial flange 6 and of at least one second sensor 202 on the distal axial wall 7 allows to detect a handling of the device which is representative of an attempt to use. Thus, if only one from among the first and the second sensor 201, 202 detects the presence of a finger but not the other sensor, then the alert is not generated. However, the activation of only one of the first and second sensors advantageously allows to “awaken” the electronic module 100, to make it go from its standby mode to its active mode.

Advantageously, the detection of an attempt to use is only validated if the sensor system detects the simultaneous activation of a first sensor 201 and of a second sensor 202 for a predetermined minimum time, for example at least one second. This allows to remove furtive contacts not representative of a desire to use the device.

Several embodiments of the sensor system are possible.

According to a first variant, the first and second sensors 201, 202 are microcurrent sensors. In this first variant, each sensor comprises a positive pole and a negative pole, and a microcurrent is generated with a finger contacts the first sensor 201 and another finger contacts the second sensor 202.

In a variant, the first and second sensors 201, 202 are capacitive sensors. In this second variant also, a so-called “clamped” handling is detected, with at least one finger on the first sensor 201 of the radial flange 6 and at least one finger on the second sensor 202 on the distal axial wall 7.

Also, according to other variants, the first and second sensors 201, 202 can be infrared sensors, luminosity sensors, audio sensors, temperature sensors or humidity sensors. Possibly, a combination of sensors is possible, the first sensor 201 not being necessarily identical to the second sensor 202.

When the sensor system detects an attempt to use, the GPS module 102 will thus establish a geographical position of the device and the GSM module 101 will make an automatic emergency call to an emergency number, such as for example 110 in Europe, 15 or 18 in France, or 911 in the USA.

In an advantageous variant, the automatic emergency call is postponed by a few seconds from detecting an attempt to use by the sensor system, for example by timeout means provided in the electronic module 100. This implementation allows to deactivate the emergency call if, in this interval, the device is actually actuated by the user. In this event, the device would comprise a third sensor capable of detecting the actuation, for example the axial movement of the body 1 with respect to the dispensing head 2.

The automatic emergency call can comprise the transmission of a pre-recorded message accompanied by the geographical position determined by the GPS module 102. The call can also comprise a code for automatically identifying the drug arranged in the device for dispensing a fluid product. Advantageously, a coding could be provided for each sensitive drug to which the present invention applies more specifically. The call can also comprise an identification of the user, as well as timestamping.

In a variant, the call can put the user in contact with the emergency service, such that the user can directly communicate with said emergency service.

According to a first variant, the GSM module 101 can make emergency calls until the battery 103 is empty.

According to a second variant, the call number can be predefined. According to a third variant, the device only makes one single call.

After the call(s), the electronic module 100 can return to standby mode or switch off.

The wireless communication module 101 could, in a variant, comprise a Wi-Fi module and/or a Bluetooth® module, and more generally, any type of wireless communication means using various bandwidths and/or frequencies.

The geographical location module 102 could, in a variant, comprise a Galileo module, and more generally any type of geographical location means. Thus, the geographical location module could not use satellite connection, but an antenna array, for example via a GSM or Wi-Fi or similar network. A combination of the two location means, by satellite and by antenna, can also be considered.

The power source 103 could, in a variant, be made in the form of a rechargeable battery. Moreover, a condenser, or any similar means, can be associated with the power source 103 to allow to deliver, if needed, a high current to the different modules described above.

The present invention is described above with reference to several advantageous embodiments, but naturally any modification could be applied thereto by a person skilled in the art, without going beyond the ambit of the present invention, as defined by the accompanying claims.

1. A device for dispensing a fluid product comprising a body and a dispensing head provided with a dispensing orifice and axially movable with respect to said body during actuation, said body receiving a vessel containing one or two doses of fluid product, said device comprising an electronic module which comprises a wireless communication module, such as a GSM module and/or a Wi-Fi module and/or a Bluetooth® module, a geographical location module, such as a GPS module and/or an antenna array, and a power source such as a battery, characterised in that said device comprises a sensor system for automatically detecting and signalling handling representative of an attempt to use said device, said sensor system comprising at least one first sensor arranged on said dispensing head and at least one second sensor arranged on said body, said sensor system detecting handling representative of an attempt to use said device when said first and second sensors are activated simultaneously, said wireless communication module being designed to make an automatic emergency call to an emergency number when said sensor system detects handling representative of an attempt to use said device.

2. The device according to claim 1, wherein said at least one first sensor is a microcurrent sensor, a capacitive sensor, an infrared sensor, a luminosity sensor, an audio sensor, a temperature sensor or a humidity sensor.

3. The device according to claim 1, wherein said at least one second sensor is a microcurrent sensor, a capacitive sensor, an infrared sensor, a luminosity sensor, an audio sensor, a temperature sensor or a humidity sensor.

4. The device according to claim 1, wherein, before actuation, said electronic module is switched off or in standby mode, with a zero or minimum energy consumption.

5. The device according to claim 4, wherein said electronic module is switched on or awakened by said sensor system to go from its standby or switched off mode to an active mode.

6. The device according to claim 1, wherein said dispensing head includes a radial flange on which the user places one or more fingers during actuation, said at least one first sensor being disposed on said radial flange.

7. The device according to claim 1, wherein said body includes a distal axial wall on which the user places one or more fingers, typically the thumb, during actuation, said at least one second sensor being disposed on said distal axial wall.

8. The device according to claim 1, wherein said electronic module comprises timeout means to postpone the automatic emergency call by a few seconds from detecting an attempt to use by the sensor system.

9. The device according to claim 1, wherein said vessel comprises a hollow tube having a proximal axial opening that is closed by a stopper that is adapted to slide in leaktight manner in said tube during actuation.

10. The device according to claim 1, wherein said dispensing head comprises a hollow cannula connected on one side to said dispensing orifice and provided on the other side with a perforator tip provided with an opening for piercing said stopper

11. The device according to claim 1, wherein said dispensing head comprises a hollow cannula connected on one side to said dispensing orifice and provided on the other side with a perforator tip provided with an opening for piercing said stopper

12. The device according to claim 1, wherein said fluid product is a powder.

13. The device according to claim 1, wherein said vessel contains one single dose of fluid product, dispensed in one single actuation.

14. The device according to claim 1, wherein said vessel contains two doses of fluid product, dispensed in two successive actuations.
